(19) United States
(12) Patent Application Publication
(10) Pub. No.: US 2020/0147254 A1
(43) Pub. Date: May 14, 2020

(54) DISINFECTION OR STERILIZATION INDICATOR

(71) Applicants: Esteban LOMBARDÍA, Pueblo Esther (AR); Adrián Jesús ROVETTO, Rosario (AR)

(72) Inventors: Esteban LOMBARDÍA, Pueblo Esther (AR); Adrián Jesús ROVETTO, Rosario (AR)

(21) Appl. No.: 16/186,286

(22) Filed: Nov. 9, 2018

(51) Int. Cl. A61L 2/28; A61L 9/14; A61L 9/015

(52) U.S. Cl. CPC A61L 2/28; A61L 2202/25; A61L 9/015; A61L 9/14

(57) ABSTRACT

An indicator device and method for monitoring disinfection or sterilization of closed environments and surfaces located in said closed environments, the disinfection and sterilization made by applying a disinfection or sterilization agent, wherein the indicator device comprises a carrier coated or embedded with substances or reagents that produce a change in color or in other readily visible property when contacting said disinfection or sterilization agent, and wherein the carrier is configured as a polyhedron, a cone, a cylinder, a sphere or any three-dimensional shape. A planar carrier having weakened linear areas or groves, printed lines or printed images, and so it is configured to be folded along said linear areas, groves or printed lines or images so as to form a polyhedron, a cone or a cylinder and so it is configured to be folded along said linear areas, groves or printed lines or images so as to form a polyhedron, a cone or a cylinder.

### FIELD OF THE INVENTION

The present invention relates to the field of disinfection or sterilization of closed environments by means of gas, plasma, steam, spray, chemical vapors or chemical fogs. More particularly, the invention relates to an indicator for monitoring such disinfection or sterilization.

### BACKGROUND OF THE INVENTION

The disinfection or sterilization of closed environments and surfaces contained therein, by means of chemical agents such as hydrogen peroxide, formaldehyde, ozone, peracetic acid and ethylene oxide, is quite recent in the industrial and medical field, especially in operating rooms of hospitals and hospitalization rooms. Since new devices designed to disinfect environmental surfaces have been launched to the market recently, said devices being capable of producing steam, chemical vapors, plasma, spray, fog or gas from chemicals, there is a need to control the sterilization efficiency of said chemical agents in different locations within the treated environment.

A wide variety of sterilization indicator strips, tapes or labels are well known in the art for monitoring the sterilization process of goods, by means of plasma, spray, fog, steam, chemical vapors or gas. This kind of indicators are generally based on a change of color or other readily visible property in the indicator resulting from a physical change or chemical reaction of suitable substances or reagents coated on or embedded in the strip, tape or label.

There is abundant patent literature regarding sterilization indicators produced during the last five or six decades.

U.S. Pat. No. 3,386,807, assigned to Johnson & Johnson and published on Jun. 4, 1968, disclosed sterilization indicator tapes coated with zinc diethyl dithiocarbamate and bismuth subgallate for monitoring sterilization with steam.

U.S. Pat. No. 3,568,627 (Selinger et al.) published on Mar. 9, 1971, disclosed indicator cards for recording and monitoring sterilization carried out in an autoclave and intended to be placed within the packaging of goods to be sterilized.

U.S. Pat. No. 4,138,216 (Larsson et al.) published on Feb. 6, 1979, disclosed a device for monitoring sterilization with ethylene oxide comprising a wick impregnated with an ethylene oxide responsive compound and sealed all but one end with an ethylene oxide impervious film.

U.S. Pat. No. 5,344,017 (Wittrock) published on Sep. 6, 1994, disclosed a pouch for sterilization of medical instruments, comprising an indicator protected between the pouch walls. Any suitable indicator for detecting steam, vapor or organic reagents like ethylene oxide may be used according to the invention.

Patent application WO 00/061200 (Patel) published on Oct. 19, 2000, disclosed layered indicators for monitoring sterilization with plasma.

Patent application WO 01/010471 (Patel) published on Feb. 15, 2001, disclosed a sterilization process with ethylene oxide monitored by a layered indicator responsive to pH rise.

None of the prior art documents teaches or suggests the volumetric monitoring of environments, like operation rooms or other hospital facilities, or surfaces therein. Then, there is a need of indicators with suitable structures and shapes that allow their location in different areas of an environment and allow assessing the sterilizing agent action in a volumetric way, e.g. determining that the sterilization agent has reached any location within the environment evidenced by the proper surrounding and contact with the indicator.

### SUMMARY OF THE INVENTION

The present invention is intended to fulfil that need and therefore it relates to indicators to control disinfection or sterilization of environments by means of steam, plasma, gas, spray, fog or vapors.

Therefore, in a first aspect, it is an object of the present invention an indicator device for monitoring disinfection or sterilization of closed environments and surfaces located in said closed environments, wherein the disinfection and sterilization are made by applying a disinfection or sterilization agent, wherein the indicator device comprises a carrier coated or embedded with substances or reagents that produce a change in color or in other readily visible property when contacting said disinfection or sterilization agent, and wherein the carrier is configured as a polyhedron, a cone, a cylinder, a sphere or other three-dimensional shape.

In preferred embodiments of this first aspect of the present invention, the disinfection or sterilization agent is steam, plasma, a vaporized, sprayed or gaseous chemical agent, preferably vaporized hydrogen peroxide, hydrogen peroxide plasma, ethylene oxide, ozone, formaldehyde, ortho-phthalaldehyde (OPA) or peracetic acid.

In a preferred embodiment of this first aspect of the present invention, the polyhedron is a tetrahedron, a cube, a pentahedron or an icosahedron.

In an embodiment of this first aspect of the present invention, the polyhedron is formed by a folded planar carrier forming dihedral angles.

In a preferred embodiment of this first aspect of the present application, the planar carrier is folded so as to form a polyhedron preferably a tetrahedron, a cube, a pyramid, a pentahedron or an icosahedron.

In another preferred embodiment of this first aspect of the present invention the carrier is configured as a cone or a sphere.

In embodiments of this first aspect of the present invention, the substances or reagents that produce a change in color or in other readily visible property when contacting said sterilization agents are selected from commercially available sterilization indicator inks.

In embodiments of this first aspect of the present invention, the sterilization indicator inks comprise Acrylic Resin, Sodium bromide and CI 74190 (Turquoise Blue) for reaction with ethylene oxide; CI 13025 (Methyl Orange), Polyvinyl alcohol and CI 74400 (Solvent blue 70) for reaction with formaldehyde; Acrylic Resin, CI 42045 (Blue Acid 1), CI 45170 (Solvent Red 49), CI 74190 (Turquoise Blue) and sodium nitrite for reaction with peracetic acid and CI 11210 (Disperse Red 17), EDTA and acrylic resin for reaction with hydrogen peroxide plasma

In preferred embodiments of this first aspect of the present invention, the carrier is made of paper, cardboard, plastics, polymeric material or metal alloys, more preferably of paper or cardboard.

In a second aspect, it is an object of the present invention a planar carrier coated or embedded with substances or reagents that produce a change in color or in other readily visible property when contacting a disinfection and sterilization agent, wherein the carrier has weakened linear areas or groves, printed lines or printed images, and so it is configured to be folded along said linear areas, groves or printed lines or images so as to form a polyhedron, a cone or a cylinder.

In embodiments of this second aspect of the present invention, the disinfection or sterilization agent is steam, plasma, vaporized hydrogen peroxide, hydrogen peroxide plasma, ethylene oxide, ozone, formaldehyde, ortho-phthalaldehyde (OPA) or peracetic acid.

In a preferred embodiment of this second aspect of the invention, the substances or reagents that produce a change in color or in other readily visible property when contacting the disinfection or sterilization agents are selected from commercially available sterilization indicator inks.

In a third aspect, it is another object of the invention a method for monitoring disinfection or sterilization of environments and surfaces located in said closed environments the disinfection and sterilization made by applying a disinfection or sterilization agent, the method comprising the steps of:
selecting the locations within an environment where it is critical or necessary to confirm the disinfection or sterilization action of a disinfection or sterilization agent;
providing said locations with indicator devices for monitoring the disinfection or sterilization, the indicator devices comprising a carrier homogeneously coated or embedded with substances or reagents that produce a change in color or in other readily visible property when contacting the disinfection or sterilization agent, wherein the carrier is configured as a polyhedron, a cone, a cylinder or a sphere;
carrying out a sterilization step, including the step of introducing the disinfection or sterilization agent into the environment; and
observing the change in color or in other visible property.

In a preferred embodiment of this third aspect of the invention, the closed environment to be sterilized is an operation room or a hospitalization room in a hospital, or a doctor's office in a clinic, a food production area or pharma production areas.

In a preferred embodiment of this third aspect of the invention, the closed environment to be sterilized is a storage room for medical instruments.

In a preferred embodiment of this third aspect of the invention, the closed environment to be sterilized belongs to food facilities, pharma and a medical devices facilities.

In a preferred embodiment of this third aspect of the invention, the disinfection or sterilization agent is steam, a vaporized chemical agent or a gaseous chemical agent, more preferably hydrogen peroxide, ozone or peracetic acid.

### BRIEF DESCRIPTION OF THE FIGURES

FIG. 1 shows an embodiment of the indicator of the present invention wherein the carrier is configured as an tetrahedron.

FIG. 2 shows an embodiment of the indicator of the present invention wherein the carrier is configured as a pyramid with a triangular base.

FIG. 3 shows an embodiment of the indicator of the present invention wherein the carrier is configured as a pyramid with an hexagonal base.

FIG. 4 shows an embodiment of the indicator of the present invention wherein the carrier is configured as a icosahedron.

FIG. 5 shows an embodiment of the indicator of the present invention wherein the carrier is planar and folded so as to form three dihedral angles, also designated as “tent”.

FIG. 6 shows an embodiment of the indicator of the present invention wherein the carrier is configured as a cube.

FIG. 7 shows an embodiment of the indicator of the present invention wherein the carrier is configured as a cone.

FIG. 8 shows an embodiment of the indicator of the present invention wherein the carrier is configured as a sphere.

FIG. 9 shows an embodiment of the indicator of the present invention wherein the carrier is configured as a cylinder.

FIGS. 10A-10D shows an embodiment of the indicator of the present invention wherein the carrier is a planar piece of paper or cardboard with weakened portions and configured to be folded to form the indicators of FIGS. 2, 4, 5 and 6, respectively.

FIG. 11 shows a layout of a room in which disinfection assays were performed.

### DETAILED DESCRIPTION OF THE INVENTION

Disinfection or sterilization of environments faces some limitations regarding the control of parameters that determine the effectiveness of disinfection or sterilization of environmental surfaces and air therein, since sterilization equipment cannot accurately control parameters like humidity, temperature, volume of objects and size of walls in the room or chamber to be sterilized. Besides that, said processes are not capable of eliminating the environment air and thus the sterilizing agent faces many obstacles for diffusing and reaching all surfaces and zones to be sterilized.

The present invention provides chemical indicators with different structures and shapes that allow their location in different areas of an environment and allow assessing the sterilizing agent action in a volumetric way, not limited only to surfaces.

Therefore, the object of the present invention is to provide a sterilization or disinfection chemical indicator that comprises substances or reagents that produce a change in color or in other readily visible property, available in the market as reactive inks, able to be responsive to specific sterilizing agents and/or physical or chemical parameters predefined for a specific sterilization process, the indicator being thus capable of confirming the sterilization of environmental surfaces and air where sterile conditions are needed.

The present invention allows verifying a disinfection or sterilization process by exposing a reactive ink on surfaces with different geometrical shapes so as to make it possible to analyze in a volumetric conception in all the possible directions the effectiveness of the sterilization process in every zone of interest within the environment to be sterilized, e.g. operation rooms in hospitals, autoclave chambers, doctor's offices in clinics, food or pharmaceutical facilities.

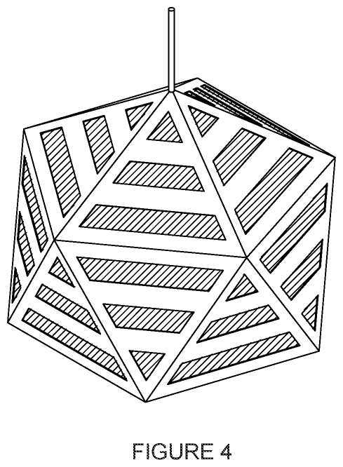

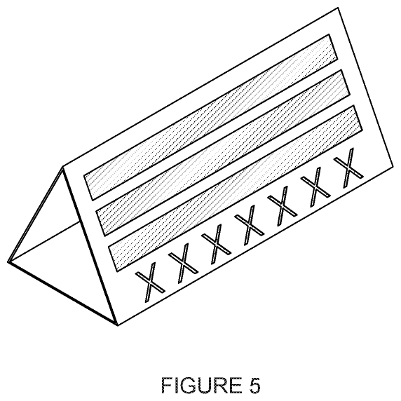

Therefore, the present invention allows selecting an appropriate indicator configuration, according to the location where the sterilization verification is needed, providing a wide variety of orientations of the carrier surfaces embedded with a reactive ink, ranging from two surfaces or faces as shown in FIG. 5 up to twenty faces as shown in FIG. 4 or more, or even a continuous multi-orientation surface in case of a carrier configured as a sphere, cone or cylinder. Thus, the indicator of the present invention provides a solution for a long time unsatisfied need, a need that the well-known planar indicators are not able to fulfill.

The indicator of the present invention may be located directly on the environmental surfaces to be sterilized or may be attached to or hanged from a wall of the room or chamber to be sterilized, e.g. at distal ends from the sterilizing agent source in a chamber or room, in order to allow the verification of the sterilization efficiency at every location of interest.

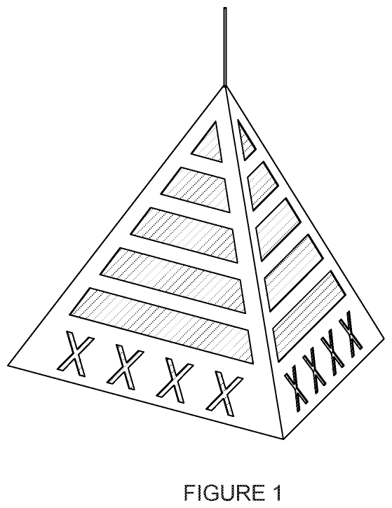

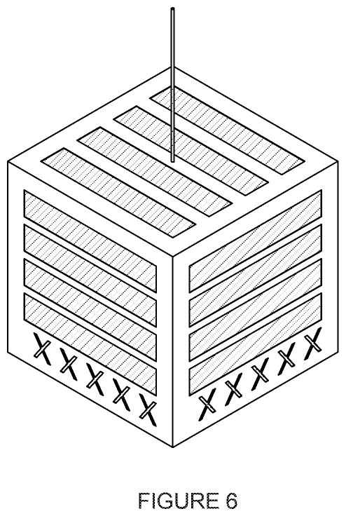

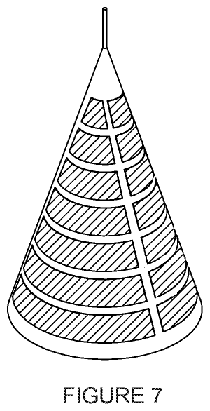

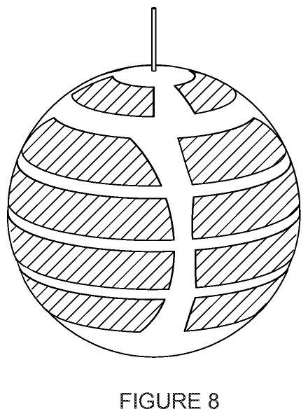

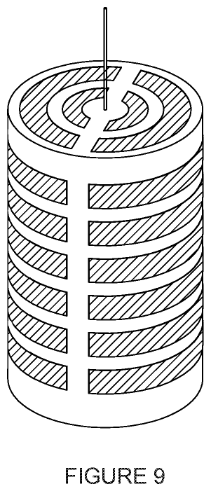

FIGS. 1 to 9 show preferred exemplary embodiments of the present invention, where the indicators are configured in different three-dimensional shapes, from one indicator having three faces up to an icosahedral indicator having twenty faces, as well as conical, cylindrical and spherical indicators. The indicators may consist of solid bodies or hollow bodies, in order to balance costs and structural properties (e.g. weight, toughness, or structural resistance). In said figures, the striped areas show the printed areas in each indicator device containing an indicator ink and the “X” symbols indicate a printed text with information about the device (e.g. trademark, ways of use or expiration date).

Indicators of FIGS. 1 to 9 may be made of a polymeric material or metal alloy as a solid body or may be formed by folding up a planar cardboard or paper carrier. The indicator devices may comprise on their top portion a piece of rope or wire for being hanged instead of being placed on top of a surface, if needed or desired.

FIG. 5 shows a three-faced indicator formed by folding up a cardboard carrier.

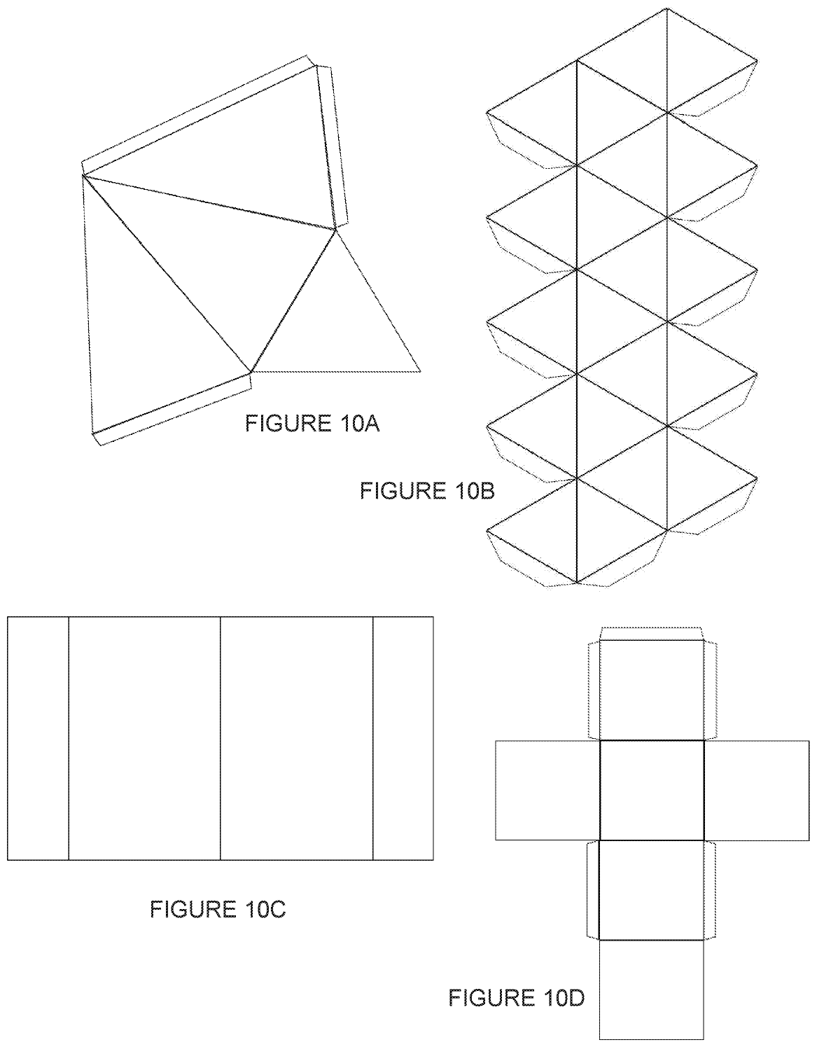

FIGS. 10A-10D show exemplary cardboard carriers shaped so as to be folded to form hollow indicators in the form of a pyramid, an icosahedron, a “tent” and a cube, according to FIGS. 1, 4, 5 and 6, respectively.

The substances or reagents that produce a change in color or in other readily visible property when contacting the steam, chemical vapors or gas, that may be applied to the indicator device of the present invention may be selected from commercially available sterilization indicator inks, preferably Acrylic Resin, Sodium bromide and CI 74190(Turquoise Blue) for reaction with ethylene oxide; CI 13025(Methyl Orange), Polyvinyl alcohol and CI 74400 (Solvent Blue 70) for reaction with formaldehyde; Acrylic Resin, CI 42045 (Blue Acid 1), CI 45170 (Solvent Red 49), CI 74190 (Turquoise Blue) and sodium nitrite for reaction with peracetic acid and CI 11210 (Disperse Red 17), EDTA and acrylic resin for reaction with hydrogen peroxide plasma.

### PREPARATION EXAMPLES

### General Process for Manufacturing Chemical Indicators

The manufacturing process begins with the design of all possible three-dimensional shapes for the product on a planar template suitable to be folded in order to generate a three-dimensional body having several faces, which are to be printed with the chemical indicator ink, so that such body may be placed in a certain area of a room, space or closed environment for the purpose of disinfection. The geometrical body, printed with the chemical indicator ink, is suitable to be either suspended from the ceiling of the room or space, hung by a hook on a wall, or placed on top of a surface.

The main object of the invention is that the indicators can challenge the most critical variables of the environmental disinfecting process. The different faces printed with the indicator ink will allow verifying whether the disinfection agent has reached every sector and corner of the room, space or closed environment. This would be easily verified by checking the color change of the indicator ink from the original color to the resulting color after disinfection.

The indicator ink consists in a chemical formulation included within a matrix or polymer that allows the adhesion or anchorage thereof onto the carrier or substrate used. The indicator ink changes its color in the presence of the sterilization or disinfection agent. This change in color depends on several critical factors affecting the action of the sterilization or disinfection agent. Exposure time, ambient temperature and humidity are factors affecting the performance of the disinfection process. Therefore, reactivity of the ink formulation is important for its change of color, indicative of the sterilization or disinfection process that is taking place.

The chemical indicator inks may be printed on a carrier sheet by serigraphy or offset printing and the sheet so printed is subsequently submitted to a die-cast process in order to produce the planar template suitable to be folded into a three-dimensional body.

In preferred embodiments of the present invention, chemical indicators consist of a synthetic piece of paper or carton, most typically rectangular in shape, which is appropriately cut to fit the printing format of the offset printer machine. Paper is printed in black ink, color ink and specific indicator ink for reaction with the intended sterilizing agents.

The printing process is preferably carried out in two steps. In a first step, the preparation of the matrices to be used in the printing system as pre-printing originals:
metallic sheets for offset machine: for pre-printing originals, the design is printed on a thermal, non-ablative, digital sheet (KODAK®, of about 605×745×0.30 mm in size), by CTP laser technology (needed laser energy 90-130 mJ/cm² using KODAK 400 xLo system). Only product-related information is printed on this sheet, such as batch, manufacturing date, expiration date, disinfection agent, etc., to be printed through the offset system.
graphical film: exemplary film specifications: emulsion-coated AGFA® film anti-static polyester base 0.10 mm, which is etched by a red Helium-Neon (He—Ne) laser and diode laser (630-670 nm). The graphical film allows printing the ink according to the designs predetermined by the system.

In a second step, when carried out in an offset machine, both paper and metallic sheet are placed inside the offset machine. By applying pressure, the etched design is transferred to the substrate. Typically, the offset printing is a printing method that consists in applying a generally oily ink on a metallic plate, i.e., the abovementioned metallic sheet, composed by an aluminum alloy. The sheet is wetted with water or a polar solution for repelling the ink at the zones with no image, so that the sheet can absorb ink in the zones where a hydrophobic or apolar compound is present (also known as oily compound) having the shape of the motif to be printed, previously etched on the sheet. The image, or text, is transferred to the surface to be printed. This is not directly accomplished but through a cylinder coated with a flexible material on its surface (a mesh), generally rubber. This material receives the image for transferring by pressure to the printed surface, in this case, a synthetic paper or Chambril paper.

When the final step is carried out by serigraphy, it is first necessary to prepare the matrix, consisting of a 94×94 cm aluminum frame in which a Sefar polyester silk is tensioned at about 23 Newton. A Pintesint photosensitive emulsion is applied to this polyester silk, covering the same and all pores. The graphical film is placed on the silk and exposed for 2 minutes to ultraviolet light under vacuum that does not allow space between the film and the frame with the emulsion. Once exposed, the film is removed and the development is carried out with water. The ultraviolet light fixes the emulsion and the virgin silk remains in the areas or black shapes that were printed on the graphic film. By washing with water the unexposed part is diluted, leaving those free parts in the silk where ink is placed on, by spreading using a rubber tool. The ink passes through the mesh in image part and is deposited in the pre-printed synthetic paper or Chambril paper.

Finally, the paper or substrate die-cast is made by means of a wooden matrix with cutting blades and marks. Wood is cut with a CNC CO₂ laser (GY-1218 SH model) having an operation tolerance of ±0.2 mm and an automatic ABM-300D CNC blade folding machine. A special laminated wood, of the non-curving type, is used for laser. Blades are DIEFLEX model, with tempered edge. On the die-cast machine, the printed paper is placed on the slide and subjected to pressure against the wooden matrix, forming the final planar template that can be folded at the time of use into a three-dimensional body.

### Example 1

According to the general manufacturing process above, a template to be folded into an indicator of FIG. 1 was printed with an indicator ink for monitoring sterilization or disinfection with ethylene oxide. The indicator ink had the composition of Table 1.

TABLE 1
Composition of an indicator ink for ethylene oxide

| Compound | % |
|---|---|
| Acrylic Resin | 57 |
| 2-Butoxyethanol | 2 |
| Castor oil | 3 |
| Propylene glycol methyl ether (1^{st} step) | 2 |
| Sodium lauryl sulfate | 0.05 |
| Sodium Polyacrylate | 1 |
| Water | 8 |
| Triethanolamine | 0.5 |
| Calcium chloride | 4 |
| Magnesium chloride | 4 |
| Sodium bromide | 5 |
| Isopropanol | 6 |
| n-propyl acetate | 4 |
| CI 74190 (Turquoise Blue) | 0.4 |
| Propylene glycol methyl ether (2^{nd} step) | 3.5 |

### Example 2

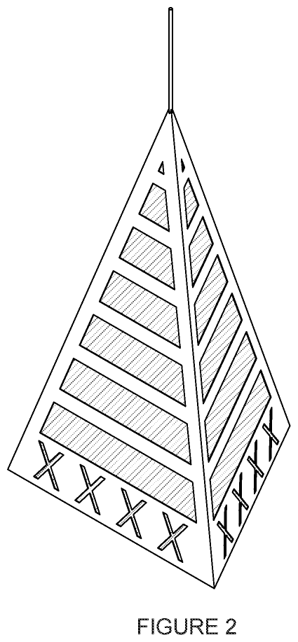

According to the general manufacturing process above, a template to be folded into an indicator of FIG. 2 was printed with an indicator ink for monitoring sterilization or disinfection with formaldehyde. The indicator ink had the composition of Table 2.

TABLE 2
Composition of an indicator ink for formaldehyde

| Compound | % |
|---|---|
| CI 13025 (Methyl Orange) | 1.3 |
| Lauric Alcohol | 2.5 |
| Water | 22 |
| Sodium Polyacrylate | 1 |
| Polyvinyl alcohol | 71 |
| Butyldiglycol | 2 |
| Propylene glycol methyl ether | 2 |
| CI 74400 (Solvent Blue 70) | 0.18 |
| Castor oil | 5 |
| 2-Butoxyethanol | 6 |

### Example 3

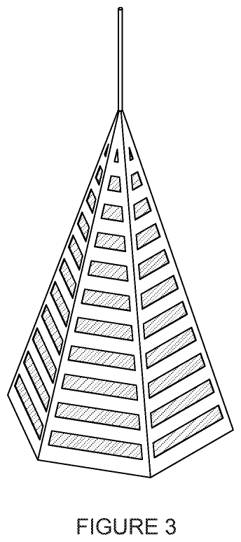

According to the general manufacturing process above, a template to be folded into an indicator of FIG. 3 was printed with an indicator ink for monitoring sterilization or disinfection with peracetic acid. The indicator ink had the composition of Table 3.

TABLE 3
Composition of an indicator ink for peracetic acid

| Compound | % |
|---|---|
| EA595- acrylic resin | 23 |
| Castor oil (1^{st} step) | 7 |
| Polyvinyl alcohol | 20 |
| Water (1^{st} step) | 7 |
| Isopropanol | 6 |
| Emulsified Dimethylpolysiloxane | 6 |
| Castor oil (2^{nd} step) | 6 |
| Ethyl acetate | 12 |
| Nitrocellulose | 1 |
| CI 42045 (Blue Acid 1) | 0.11 |
| CI 45170 (Solvent Red 49) | 0.6 |
| CI 74190 (Turquoise Blue) | 0.14 |
| Sodium nitrite | 2.9 |
| Water (2^{nd} step) | 10 |
| Ethanol | 3 |
| Propylene glycol methyl ether | 3 |

### Example 4

According to the general manufacturing process above, a template to be folded into an indicator of FIG. 4 was printed with an indicator ink for monitoring sterilization or disinfection with hydrogen peroxide plasma. The indicator ink had the composition of Table 4.

TABLE 4
Composition of an indicator ink for hydrogen peroxide plasma

| Compound | % |
|---|---|
| CI 11210 (Disperse Red 17) | 0.15 |
| Ethanol (1^{st} step) | 15 |
| Propylene glycol methyl ether | 8 |
| CI 42045 (AA1) | 0.4 |
| CI 12755 (Indapol Yellow) | 0.1 |
| Water | 6 |
| Acrylic resin | 52 |
| EDTA | 2.3 |
| Emulsified Dimethylpolysiloxane | 4 |
| Butyldiglycol | 2 |
| Acrylic Acid | 1 |
| Ethanol (2^{nd} step) | 4 |
| n-butyl acetate | 8 |

### Disinfection Monitoring Assays with the Indicators of the Invention

The indicators of the present invention were assayed in order to determine their efficacy for monitoring sterilization or disinfection processes in all areas of a closed environment.

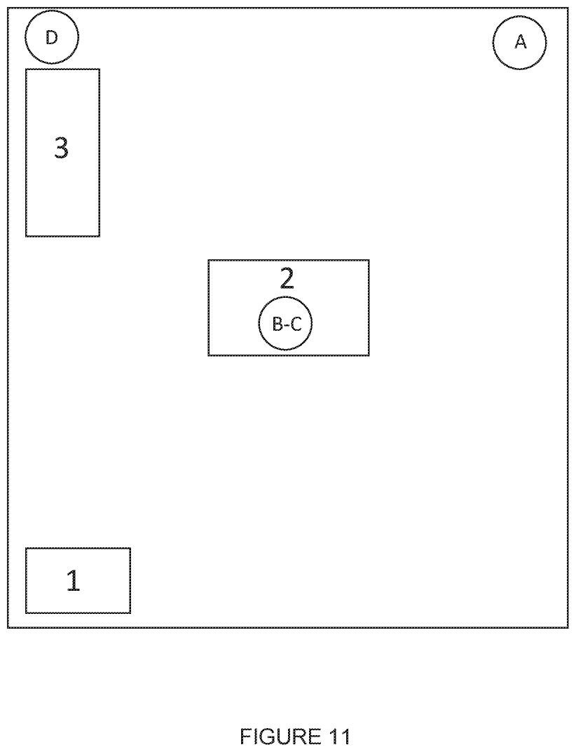

The assays were carried out in a 144 m³ room as illustrated in FIG. 11 provided with an air conditioning system capable of controlling and modifying temperature and relative humidity. A disinfection equipment 1 that provided vaporization of hydrogen peroxide solution at 1 ml/m³ was used in the assays. The furniture in the room consisted of a desk 2 and a cupboard 3.

Some disinfection process critical variables, that may lead to an unsuccessful disinfection, were tested in order to verify their effect on the process, as follows:
Solution concentration: hydrogen peroxide concentration in the solution to be vaporized;
Exposure time: amount of time between the end of vaporization step and room ventilation;
Temperature: initial room temperature;
Humidity: initial ambient humidity prior to the disinfection process; and
Set volume: operational volume as set in the disinfection equipment.

A group of indicators to be assayed were located in the positions indicated in FIG. 11, as follows:
A.—Opposite corner to the disinfection equipment 1, at 1.5 m height, with one of the indicator faces facing said corner, at 15 cm from each corner wall.
B.—Under the desk 2 in the center of the room and hanging from the desk at 65 cm height.
C.—In the center of the room, hanging from the ceiling at 2.5 m height.
D.—Between the cupboard 3 and the walls in the corner adjacent to the disinfection equipment 1, at 1.5 m height and 15 cm from each wall.

The assayed indicators were as follows:
Regular tetrahedron as shown in FIG. 1 with 10 cm edges, designated as “Pyramid”
Regular icosahedron as shown in FIG. 4 with 5 cm edges, designated as “Icosahedron”
“Trihedron” as shown in FIG. 5 with 2 faces of 10 cm wide×5 cm height and a 5 cm base, designated as “Tent”.

The assays results from optical inspection of the indicators after the disinfection process were allocated as:
“Successful”: the chemical indicator color shift reached its final color or “end point”;
“Indefinite”: the final color shift was difficult to be assessed;
“Failure”: the chemical indicator color shift did not reach the expected final color in a noticeable and/or homogeneous way; and
“N/A”: no applicable face due to the particular three-dimensional shape of the indicator.

The spatial reference made to the indicator faces are intended to have the following meaning:
“Front faces”: indicator areas facing the disinfection equipment 1;
“Side faces”, “Top faces” and “Bottom faces”: when appropriate, indicator areas not directly facing the disinfection equipment 1; and
“Rear faces”: indicator areas facing away from the disinfection equipment 1.

The disinfection results are summarized in the following tables:

TABLE 5
“End point” (Successful) Assays
Assay conditions

| Solution | Exposure | | Ambient | Set |
|---|---|---|---|---|
| concentration | time | Temperature | humidity | Volume |
| 35% | 30 minutes | 25° C. | 62% | 144 m³ |

End point assay results

| | | Front | Rear | Side | Top | Bottom |
|---|---|---|---|---|---|---|
| Indicator | Position | faces | faces | faces | faces | faces |
| Pyramid | A | Successful | Successful | N/A | N/A | Successful |
| | B | Successful | Successful | N/A | N/A | Successful |
| | C | Successful | Successful | N/A | N/A | Successful |
| | D | Successful | Successful | N/A | N/A | Successful |
| Icosahedron | A | Successful | Successful | Successful | Successful | Successful |
| | B | Successful | Successful | Successful | Successful | Successful |
| | C | Successful | Successful | Successful | Successful | Successful |
| | D | Successful | Successful | Successful | Successful | Successful |
| Tent | A | Successful | Successful | N/A | N/A | N/A |
| | B | Successful | Successful | N/A | N/A | N/A |
| | C | Successful | Successful | N/A | N/A | N/A |
| | D | Successful | Successful | N/A | N/A | N/A |

TABLE 6
Concentration Failure Assays
Assay conditions

| Solution | Exposure | | Ambient | Set |
|---|---|---|---|---|
| concentration | time | Temperature | humidity | Volume |
| 3% | 30 minutes | 25° C. | 61% | 144 m³ |

End point assay results

| | | Front | Rear | Side | Top | Bottom |
|---|---|---|---|---|---|---|
| Indicator | Position | faces | faces | faces | faces | faces |
| Pyramid | A | Indefinite | Failure | N/A | N/A | Failure |
| | B | Indefinite | Failure | N/A | N/A | Failure |
| | C | Failure | Failure | N/A | N/A | Indefinite |
| | D | Failure | Failure | N/A | N/A | Failure |
| Icosahedron | A | Indefinite | Failure | Failure | Indefinite | Indefinite |
| | B | Successful | Failure | Indefinite | Failure | Indefinite |
| | C | Indefinite | Failure | Failure | Failure | Indefinite |
| | D | Failure | Failure | Failure | Failure | Failure |
| Tent | A | Indefinite | Failure | N/A | N/A | N/A |
| | B | Successful | Failure | N/A | N/A | N/A |
| | C | Indefinite | Failure | N/A | N/A | N/A |
| | D | Failure | Failure | N/A | N/A | N/A |

TABLE 7
Time Failure Assays
Assay conditions

| Solution | Exposure | | Ambient | Set |
|---|---|---|---|---|
| concentration | time | Temperature | humidity | Volume |
| 35% | 0 minutes | 25° C. | 62% | 144 m3 |

End point assay results

| | | Front | Rear | Side | Top | Bottom |
|---|---|---|---|---|---|---|
| Indicator | Position | faces | faces | faces | faces | faces |
| Pyramid | A | Failure | Failure | N/A | N/A | Failure |
| | B | Indefinite | Failure | N/A | N/A | Failure |
| | C | Indefinite | Failure | N/A | N/A | Indefinite |
| | D | Failure | Failure | N/A | N/A | Failure |
| Icosahedron | A | Indefinite | Failure | Failure | Failure | Failure |
| | B | Successful | Failure | Indefinite | Failure | Indefinite |
| | C | Indefinite | Failure | Failure | Failure | Indefinite |
| | D | Failure | Failure | Failure | Failure | Failure |
| Tent | A | Failure | Failure | N/A | N/A | N/A |
| | B | Successful | Failure | N/A | N/A | N/A |
| | C | Indefinite | Failure | N/A | N/A | N/A |
| | D | Failure | Failure | N/A | N/A | N/A |

TABLE 8
Volume Failure Assays
Assay conditions

| Solution | Exposure | | Ambient | Set |
|---|---|---|---|---|
| concentration | time | Temperature | humidity | Volume |
| 35% | 30 minutes | 26° C. | 61% | 20 m³ |

End point assay results

| | | Front | Rear | Side | Top | Bottom |
|---|---|---|---|---|---|---|
| Indicator | Position | faces | faces | faces | faces | faces |
| Pyramid | A | Indefinite | Failure | N/A | N/A | Failure |
| | B | Indefinite | Failure | N/A | N/A | Failure |
| | C | Failure | Failure | N/A | N/A | Failure |
| | D | Failure | Failure | N/A | N/A | Failure |
| Icosahedron | A | Indefinite | Failure | Failure | Indefinite | Indefinite |
| | B | Successful | Failure | Indefinite | Failure | Indefinite |
| | C | Successful | Failure | Failure | Failure | Failure |
| | D | Failure | Failure | Failure | Failure | Failure |
| Tent | A | Failure | Failure | N/A | N/A | N/A |
| | B | Successful | Failure | N/A | N/A | N/A |
| | C | Indefinite | Failure | N/A | N/A | N/A |
| | D | Failure | Failure | N/A | N/A | N/A |

TABLE 9
Temperature Failure Assays
Assay conditions

| Solution | Exposure | | Ambient | Set |
|---|---|---|---|---|
| concentration | time | Temperature | humidity | Volume |
| 35% | 30 minutes | 10° C. | 62% | 144 m3 |

End point assay results

| | | Front | Rear | Side | Top | Bottom |
|---|---|---|---|---|---|---|
| Indicator | Position | faces | faces | faces | faces | faces |
| Pyramid | A | Indefinite | Failure | N/A | N/A | Failure |
| | B | Indefinite | Failure | N/A | N/A | Failure |
| | C | Indefinite | Failure | N/A | N/A | Indefinite |
| | D | Failure | Failure | N/A | N/A | Failure |
| Icosahedron | A | Indefinite | Failure | Indefinite | Indefinite | Indefinite |
| | B | Successful | Failure | Indefinite | Failure | Indefinite |
| | C | Indefinite | Failure | Indefinite | Failure | Indefinite |
| | D | Indefinite | Failure | Failure | Failure | Failure |
| Tent | A | Indefinite | Failure | N/A | N/A | N/A |
| | B | Indefinite | Failure | N/A | N/A | N/A |
| | C | Indefinite | Failure | N/A | N/A | N/A |
| | D | Failure | Failure | N/A | N/A | N/A |

TABLE 10
Humidity Failure Assays (high)
Assay conditions

| Solution | Exposure | | Ambient | Set |
|---|---|---|---|---|
| concentration | time | Temperature | humidity | Volume |
| 35% | 30 minutes | 26° C. | 90% | 144 m3 |

End point assay results

| | | Front | Rear | Side | Top | Bottom |
|---|---|---|---|---|---|---|
| Indicator | Position | faces | faces | faces | faces | faces |
| Pyramid | A | Failure | Failure | N/A | N/A | Failure |
| | B | Indefinite | Failure | N/A | N/A | Failure |
| | C | Failure | Failure | N/A | N/A | Failure |
| | D | Failure | Failure | N/A | N/A | Failure |
| Icosahedron | A | Failure | Failure | Failure | Failure | Failure |
| | B | Indefinite | Failure | Indefinite | Failure | Failure |
| | C | Indefinite | Failure | Failure | Failure | Indefinite |
| | D | Failure | Failure | Failure | Failure | Failure |
| Tent | A | Failure | Failure | N/A | N/A | N/A |
| | B | Indefinite | Failure | N/A | N/A | N/A |
| | C | Indefinite | Failure | N/A | N/A | N/A |
| | D | Failure | Failure | N/A | N/A | N/A |

TABLE 11
Summary of Assays with Failure Results
Assay conditions

| | End | Concentration | Time | Volume | Temperature | Humidity |
|---|---|---|---|---|---|---|
| | Point | Failure | Failure | Failure | Failure | Failure |
| Solution Concentration | 35% | 3% | 35% | 35% | 35% | 35% |
| Exposure Time | 30 min. | 30 min. | 0 min. | 30 min. | 30 min. | 30 min. |
| Temperature | 26° C. | 25° C. | 25° C. | 26° C. | 10° C. | 26° C. |
| Ambient Humidity | 62% | 61% | 62% | 61% | 62% | 90% |
| Set Volume | 144 m³ | 144 m³ | 144 m³ | 20 m³ | 144 m³ | 144 m³ |

End point assay results

| | | | Concentration | Time | Volume | Temperature | Humidity |
|---|---|---|---|---|---|---|---|
| Indicator | Position | End point | Failure | Failure | Failure | Failure | Failure |
| Pyramid | A | Successful | Failure | Failure | Failure | Failure | Failure |
| | B | Successful | Failure | Failure | Failure | Failure | Failure |
| | C | Successful | Failure | Failure | Failure | Failure | Failure |
| | D | Successful | Failure | Failure | Failure | Failure | Failure |
| Icosahedron | A | Successful | Failure | Failure | Failure | Failure | Failure |
| | B | Successful | Failure | Failure | Failure | Failure | Failure |
| | C | Successful | Failure | Failure | Failure | Failure | Failure |
| | D | Successful | Failure | Failure | Failure | Failure | Failure |
| Tent | A | Successful | Failure | Failure | Failure | Failure | Failure |
| | B | Successful | Failure | Failure | Failure | Failure | Failure |
| | C | Successful | Failure | Failure | Failure | Failure | Failure |
| | D | Successful | Failure | Failure | Failure | Failure | Failure |

The assay results of Tables 1-11 show clearly that even in those cases where the Front Faces, i.e. the indicator areas facing the disinfection device, showed a successful or indefinite disinfection result, the partial or total absence of color shift in any of the Side Faces, Rear Faces or Top/Bottom Faces indicated that the disinfection process was not completely effective and some areas of the room were not effectively exposed to the disinfection agent. This is an advantageous feature of the present invention that cannot be achieved with planar indicator devices of the prior art.

While the invention has been illustrated and described in detail in the drawings and foregoing description, such illustration and description are to be considered illustrative or exemplary and not restrictive. The invention is not limited to the disclosed embodiments. Variations to the disclosed embodiments can be understood and effected by those skilled in the art in practicing the claimed invention, from a study of the drawings, the disclosure and the appended claims.

We claim:

1. An indicator device for monitoring disinfection or sterilization of closed environments and surfaces located in said closed environments, the disinfection and sterilization made by applying a disinfection or sterilization agent, wherein the indicator device comprises a carrier coated or embedded with substances or reagents that produce a change in color or in other readily visible property when contacting said disinfection or sterilization agent, and wherein the carrier is configured as a polyhedron, a cone, a cylinder or a sphere.

2. The indicator device of claim 1, wherein the disinfection or sterilization agent is steam, plasma, a vaporized, sprayed or gaseous chemical agent.

3. The indicator device of claim 2, wherein the disinfection or sterilization agent is vaporized hydrogen peroxide, hydrogen peroxide plasma, ethylene oxide, ozone, formaldehyde, orthophthalaldehide (OPA) or peracetic acid.

4. The indicator device of claim 1, wherein the polyhedron is formed by a folded planar carrier forming dihedral angles.

5. The indicator device of claim 1, wherein the cone or the cylinder is a folded planar carrier.

6. The indicator device of claim 1, wherein the polyhedron is a tetrahedron, a cube, a pentahedron, an icosahedron or a pyramid.

7. The indicator device of claim 1, wherein the carrier is configured as a cone, as a sphere or as a cylinder.

8. The indicator device of claim 1, wherein the substances or reagents that produce a change in color or in other readily visible property when contacting the disinfection or sterilization agent are sterilization indicator inks.

9. The indicator device of claim 1, wherein the carrier is made of paper, cardboard, a polymeric, plastic, synthetic material or metal alloys.

10. A planar carrier coated or embedded with substances or reagents that produce a change in color or in other readily visible property when contacting a disinfection or sterilization agent, wherein the carrier has weakened linear areas or groves, printed lines or printed images, and so it is configured to be folded along said linear areas, groves or printed lines or images so as to form a polyhedron, a cone or a cylinder.

11. The planar carrier of claim 10, wherein the carrier is configured to be folded so as to form a tetrahedron, a cube, a pentahedron, an icosahedron or a pyramid.

12. The planar carrier of claim 10, wherein the carrier is configured to be folded so as to form a cone or a cylinder.

13. The planar carrier of claim 10, wherein the disinfection or sterilization agent is selected from steam, plasma, vaporized or sprayed hydrogen peroxide, hydrogen peroxide plasma, ethylene oxide, ozone, formaldehyde, orthophthalaldehide (OPA) or peracetic acid.

14. The planar carrier of claim 10, wherein the substances or reagents that produce a change in color or in other readily visible property when contacting the disinfection or sterilization agent are sterilization indicator inks.

15. A method for monitoring disinfection or sterilization of closed environments and surfaces located in said closed environments, the disinfection and sterilization made by applying a disinfection or sterilization agent, the method comprising the steps of:
selecting the locations within a closed environment where it is critical or necessary to confirm the disinfection or sterilization action of a disinfection or sterilization agent;
providing said locations with indicator devices for monitoring the disinfection or sterilization, the indicator devices comprising a carrier coated or embedded with substances or reagents that produce a change in color or in other readily visible property when contacting the disinfection or sterilization agent, wherein the carrier is configured as a polyhedron, a cone, a cylinder or a sphere;
carrying out a sterilization step, including the step of introducing the disinfection or sterilization agent into the environment; and
observing the change in color or in other visible property.

16. The method of claim 15, wherein the closed environment to be sterilized is an operation room or a hospitalization room in a hospital, or a doctor's office in a clinic or industrial facilities.

17. The method of claim 15, wherein the closed environment to be sterilized is a storage room for medical instruments.

18. The method of claim 15, wherein the closed environment to be sterilized belongs to food facilities, pharma and medical devices industries.

19. The method of claim 15, wherein the disinfection or sterilization agent is selected from steam, plasma, vaporized hydrogen peroxide, hydrogen peroxide plasma, ethylene oxide, ozone, formaldehyde, orthophthalaldehide (OPA) or peracetic acid.

20. The method of claim 15, wherein the polyhedron is a tetrahedron, a cube, a pentahedron, an icosahedron or a pyramid.
